(19) United States
(12) Patent Application Publication
(10) Pub. No.: US 2024/0423813 A1
(43) Pub. Date: Dec. 26, 2024

(54) IMPLANT FUSION DEVICE AND METHOD OF MANUFACTURING

(71) Applicant: Spectrum Spine IP Holdings, LLC, Inlet Beach, FL (US)

(72) Inventor: James C. Robinson, Inlet Beach, FL (US)

(21) Appl. No.: 18/488,575

(22) Filed: Oct. 17, 2023

Related U.S. Application Data
(63) Continuation-in-part of application No. 18/339,577, filed on Jun. 22, 2023.
(60) Provisional application No. 63/354,748, filed on Jun. 23, 2022.

(51) Int. Cl. A61F 2/44; A61F 2/30

(52) U.S. Cl. CPC A61F 2/447; A61F 2/30942; A61F 2002/30028; A61F 2002/3008; A61F 2002/30153; A61F 2002/30154; A61F 2002/30158; A61F 2002/30789; A61F 2002/30985

(57) ABSTRACT

The present invention relates to an implant fusion device and a method of manufacturing an implant fusion device. More particularly an orthopedic or spinal implant configured to be implanted between adjacent vertebrae or within a gap in a bone or between bones, the device having a manufactured body structure simulating the physical characteristics of trabecular bone, but with improved osteoinductive features on the exterior surface wherein the device is fabricated using 3D printing. Alternatively, the implant may be made through 3D printing in a manner that results in a relatively or completely solid structure, but with a surface that mimics trabecular bone structure.

### RELATED APPLICATIONS

The present invention is a continuation in part of co-pending U.S. application Ser. No. 18/339,577 entitled “Implant Fusion Device And Method Of Manufacturing” filed on Feb. 28, 2020.

### FIELD OF THE INVENTION

The present invention relates to an implant fusion device and a method of manufacturing an implant fusion device. More particularly an orthopedic or dental implant or fastener configured to be implanted between adjacent vertebrae or within a gap in a bone or between bones, the device having improved osteoinductive features on exterior surfaces wherein the device is fabricated using 3D printing. This invention may be applicable to any implant device that contacts or interacts with bone.

### BACKGROUND OF THE INVENTION

Surgical implantation of interbody cages is typically used to provide support along the spinal column in cases where a portion of the patient's intervertebral anatomy has become weakened, diseased, or destroyed. Such support systems are also commonly used following a discectomy, where an intervertebral disc is surgically removed.

Most commonly, existing support systems typically operate by inhibiting normal movement between the adjacent vertebrae, thereby stabilizing these vertebrae at fixed positions relative to one another, with the mechanical body of the supporting structure providing the needed support along the patient's spinal column. Such supporting systems are typically made of stainless steel, titanium, titanium alloy, polymer (e.g., an organic polymer thermoplastic such as polyether ether ketone (PEEK)), carbon fiber, or ceramic and they are designed to permanently remain within the patient's body.

It is beneficial, in addition to fixation, to try to stimulate bone growth between the adjacent vertebrae. To do so, spine surgeons often use bone graft material in addition to fixation devices. Bone graft doesn't heal or fuse the spine immediately; instead, bone graft provides a foundation or scaffold for the patient's body to grow new bone. Bone graft can stimulate new bone production. When new bone grows and solidifies, fusion occurs. Although instrumentation (e.g., screws, rods) is often used for initial stabilization (post-operative), it is the healing of bone that welds vertebrae together to create long-term stability. There are two general types of bone grafts: real bone and bone graft substitutes. Real bone can come from the patient (autograft) or from a donor bone (allograft). Also used in these types of surgery are bone substitute, osteoinductive agents, stem cell products, bone morphogenic proteins, and bone cement.

There is a need for improved systems and methods for spinal fusion devices and other implant devices that interact with bone. Ideally, the spinal fusion implant device has features that facilitate new bone growth to achieve fusion of the adjacent vertebrae.

### Definitions

As used herein and in the claims:

3D printing, or additive manufacturing, is the construction of a three-dimensional object from a CAD model or a digital 3D model. The term “3D printing” can refer to a variety of processes in which material is deposited, joined or solidified under computer control to create a three-dimensional object, with material being added together, typically layer by layer.

Laser modifications can include laser etching, engraving, or any other use of a laser to change or treat a surface of an implant device or any other surface.

Nanotechnology is the engineering of functional systems at the nanometer scale. This covers both current work and concepts that are more advanced. In its original sense, nanotechnology refers to the projected ability to construct items from the bottom up, using techniques and tools being developed today to make complete, high-performance products. Alternatively, nanostructure may be developed through subtractive processes.

Cortical or compact bone can be distinguished macroscopically from cancellous or trabecular bone. Cortical bone is a dense tissue that contains less than 10% soft tissue. Cancellous or spongy bone is made up of trabeculae, shaped as interconnected plates or rods and arced structures interspersed between voids in the mineral structure that contain blood cells in the marrow space which represents more than 75% of the cancellous bone volume.

Microtechnology/Laser Micro Machining: Although similar in concept to traditional machining operations, laser micro machining (laser micromachining) is capable of creating extremely small features—generally under 1 mm, and in some cases only a few microns in size—with a high degree of repeatability and without causing significant structural damage to the surrounding material.

Micron (μm) Microns, also known as micrometers (represented as μm) are a length of measurement equal to one millionth of a meter. (1,000 μm is equal to 1 mm.)

Nanotechnology: One nanometer (nm) is one billionth, or 10-9, of a meter. By comparison, typical carbon-carbon bond lengths, or the spacing between these atoms in a molecule, are in the range 0.12-0.15 nm, and a DNA double-helix has a diameter around 2 nm. On the other hand, the smallest cellular life-forms, the bacteria of the genus Mycoplasma, are around 200 nm in length. By convention, nanotechnology is taken as the scale range 1 to 100 nm following the definition used by the National Nanotechnology Initiative in the US. The lower limit is set by the size of atoms (hydrogen has the smallest atoms, which are approximately a quarter of a nm kinetic diameter) since nanotechnology must build its devices from atoms and molecules. The upper limit is more or less arbitrary but is around the size below which the phenomena not observed in larger structures start to become apparent and can be made use of in the nano device. These new phenomena make nanotechnology distinct from devices which are merely miniaturized versions of an equivalent macroscopic device; such devices are on a larger scale and come under the description of microtechnology.

Trabecular bone is a highly porous (typically 75-95%) form of bone tissue that is organized into a network of interconnected rods and plates and arcs called trabeculae which surround pores that are filled with cellular bone marrow.

### SUMMARY OF THE INVENTION

The present invention according to a first embodiment is a method of making a spinal implant fusion device having the steps of: fabricating an implant body structure using 3D printing to create the implant body structure; additively building the body structure having a superior load bearing surface and an inferior load bearing surface and a wall structure; and wherein the body structure has at least a portion of the body structure having a plurality of interconnected struts forming porous walls with openings or passages extending inwardly from an exterior surface to a depth of 1.0 mm or greater forming a porous portion with a void volume to solid mass volume mimicking trabecular bone. Alternatively, the 3D printed structure may be completely or substantially solid with a surface structure comprised of the interconnected arcs that are raised, or created like troughs that appear to be cut into the surface but were created through 3D printing.

The average or nominal ratio of void volume to mass volume in the porous portion is in the range of 65 percent or more, preferably 75 percent replicating that of trabecular bone in an adult male. The struts of the porous walls are curved or arch shaped with openings communicating with adjacent walls. The porous portion of the implant body structure extends at least partially across the implant body structure exterior surfaces forming conduits for fluid passage throughout the device. The curved or arch shaped struts of the walls create a load bearing capacity to withstand vertical loads without collapsing. The implant fusion device has the superior load bearing surface and the inferior load bearing surface having nano nanometer-scale features resulting from laser etching. In addition to load bearing surfaces, other surfaces of an implant may be laser etched as well. The etching created through a subtractive laser process. Alternatively, the 3D printed structure may be solid or relatively solid with a 3D printed surface structure that mimics trabecular bone structure as described above, with a laser etched subtractive process then applied that results in a nanotechnology level of surface which is biologically active for the induction of bone formation and growth.

In a second embodiment, a method of making a spinal implant fusion device has the steps of: providing a 3D printed implant body structure; and subsequent subtractive laser etching which results in nanometer-level structure on at least a portion of a surface or surfaces of the implant body structure, the nanometer structure creating new bone growth attachment features to enhance osteoinductivity of the spinal implant or orthopedic fusion device.

The laser etched nanometer structural features are made into a network of features in either a random pattern or an organized pattern. The laser etching is formed by emitting laser beams unobstructed to the surfaces of the implant. The method of making a spinal implant fusion device or other orthopedic or bone implant further has the step of moving a laser about the implant body structure to create the network of features or the method has the step of moving the implant body structure about a laser to create the network of features.

The present invention also has the combination of 3D printing and laser etching in a method of making a spinal implant fusion device or orthopedic or bone device having the steps of: fabricating an implant body structure using 3D printing to create the implant body structure; additively building the body structure having a superior load bearing surface and an inferior load bearing surface and a wall structure; wherein the body structure has at least a portion of the body structure having a plurality of interconnected struts forming porous walls with openings extending inwardly from an exterior surface to a depth of 1.0 mm or greater forming a porous portion with a void volume to solid mass volume mimicking trabecular bone; and laser etching nano channels on at least a portion of the exterior surface or surfaces of the implant body structure, the nano channels creating new bone growth attachment features to enhance osteoinductivity of the spinal implant fusion device.

The method of making a spinal implant device or orthopedic device or bone implant device wherein the structure is produced through a 3D printing additive process, which is then further processed with a laser etching technology that results in a nanotechnology structure at the surface that facilitates bone attachment and growth. The 3D printing additive process creates a structure at the implant surface that mimics trabecular bone structure.

The spinal implant device or orthopedic device or bone implant device can be produced through a 3D printing additive process in a biocompatible material or materials that is further processed through a subtractive laser etching process that results in a surface or surfaces with nanometer-level structural elements. The 3D printing additive process results in surface features that mimic trabecular bone structure.

### BRIEF DESCRIPTION OF THE DRAWINGS

The invention will be described by way of example and with reference to the accompanying drawings in which:

FIG. 1 is an exemplary embodiment of the spinal implant fusion device of the present invention.

FIG. 2 is a cross-sectional view of the implant device taken along lines 2-2 of FIG. 1.

FIG. 3 is an enlarged view of a portion of an exterior surface of the present invention depicting the surface, the surface being undulating with protruding and depressed features at the surface mimicking trabecular bone.

FIG. 4 is an enlarged view of the surface showing laser etched nano channels.

FIG. 5 is an exemplary 3D printer.

FIG. 6 is an exemplary laser etching machine.

FIG. 7 is a simplified schematic outline of a portion of an exterior surface showing projections and channels or troughs of an alternative embodiment.

FIG. 8A is a simplified schematic drawing showing the making of laser etched channels using a moving laser machine.

FIG. 8B is a simplified schematic drawing showing the making of laser etched channels using a fixed laser machine with the implant being moved.

FIG. 9 is a perspective view of an expandable spinal implant device made according to the present invention.

FIG. 10 is an enlarged view of a surface made using the process of the present invention.

FIG. 11 is a magnification of the surface made using the process of the present invention.

FIG. 12 is a plan view of an exemplary bone fastener or screw.

FIG. 13 is a plan view of a second exemplary bone fastener or screw.

FIG. 14 is a view of a third exemplary bone fastener or screw.

FIG. 15 is a view of an exemplary orthopedic implant specifically a hip joint implant.

FIG. 16 is a view of an exemplary knee replacement implant.

FIG. 17 is a view of an exemplary dental implant.

FIG. 18 is a view of an exemplary treatment for the unthreaded portion of a screw.

### DETAILED DESCRIPTION OF THE INVENTION

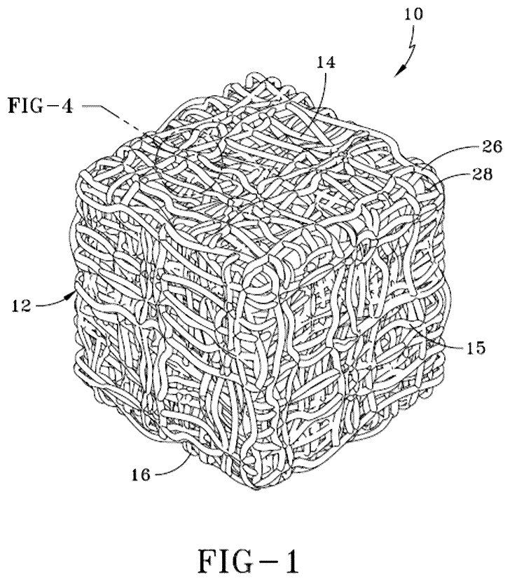

With reference to FIGS. 1-4 and 7, various views of the implant fusion device of the present invention are shown.

As shown in FIG. 1, the exemplary embodiment is shown as a cube having six sides. The configuration of the implant device 10 as illustrated has a first or superior surface 14, a second or inferior surface 16 and side surfaces 15 that surround and form the exterior surfaces of the implant body structure 12. The first surface 14 and second surface 16 provide the implant fusion device 10 with surfaces that upon implantation between two adjacent vertebral bodies will support the bone structure of the adjacent vertebral bodies. These first and second surfaces 12, 16 are in direct contact with the bone structure of the adjacent vertebral bodies of the patient upon implantation of the device 10 for a procedure where an implant fusion device is being implanted to correct a degenerative condition or other condition in a patient.

As shown, the exemplary embodiment is merely example of configurations that can be employed to make the present invention. Any number of shapes can be used in this configuration and can be any number of polygonal shapes of various shapes and sizes as long as they are sufficient to support the load between the adjacent vertebral bodies to make a proper implant fusion device.

For example, the cube shape in FIG. 1 could also be rectangular, oblong or elongated. A cylindrical device with a circular side can be used. Similarly, the device can have a pentagonal or hexagonal shape where the sides are not circular. Any number of these alternative configurations can be employed using the implant device of the present invention as will be discussed later.

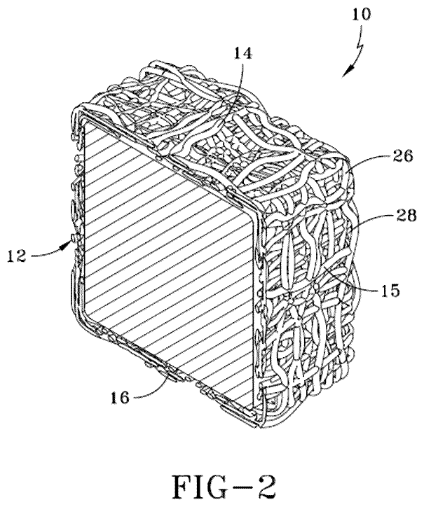

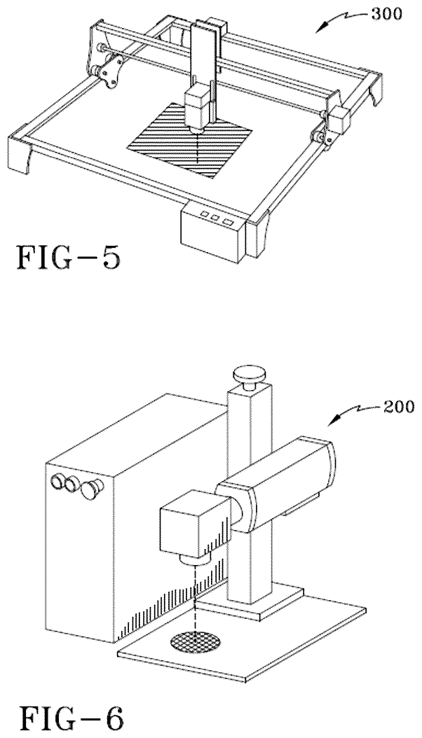

With reference to FIG. 2, a cross-sectional view is taken from FIG. 1. The cross-sectional view shows the interior structure of the implant device 10 of the present invention. As shown, the body structure 12 of the implant device 10 of the present invention has a solid central area or region and an external region with ratio of void volume to mass volume that is relatively high and replicates trabecular bone, more particularly cancellous trabecular bone wherein the high porosity creates open pathways for fluid to move in and out similar to what happens in natural bone. The body structure 12 of the implant device 10 is formed by 3D or additive printing forming exterior portions made with a plurality of interconnected struts 26, the struts 26 being curved or arched and spaced between connections with openings 28 forming a porous wall having a porosity that replicates that of an adult male or female depending on the implant being produced. An exemplary 3D printer 300 is shown in FIG. 5. It is important to note that this high ratio of porosity is extended towards a central region of the device 10. As the device extends from the perimeter to the exterior surfaces 14, 15, 16 of the implant device 10 this ratio of void volume to mass volume can be reduced dramatically, this occurs as the 3D building of the device is being performed. As such, the exterior surfaces 14, 15, 16 can have the porous walls extend approximately 1 mm or greater into the interior from the exterior surface with a center portion of the body structure having a much reduced ratio of void volume to mass volume. This reduced ratio is more tightly compacted creating a core inside the implant device with a porous structure around the entire implant device 10. This enhances the structural strength of the device 10 and provides a superior bone generating exterior surface or surfaces of the more open porosity with the interior core of the body structure 12 providing high strength.

Optionally, this porous structure of interconnected struts 26 can be made to extend throughout the implant body structure if so desired. In practice, it has been found that the depth of the surfaces mimicking the trabecular bone of at least 1 mm in depth is ideal for new bone formation and therefore the 3D manufacturing of the implant can be made simpler and less expensively by limiting the depth to 1 mm or greater. Additionally, the superior 14 and inferior 16 surface should have the porous trabecular features, but the side walls could be solid as an optional way to manufacture the implant.

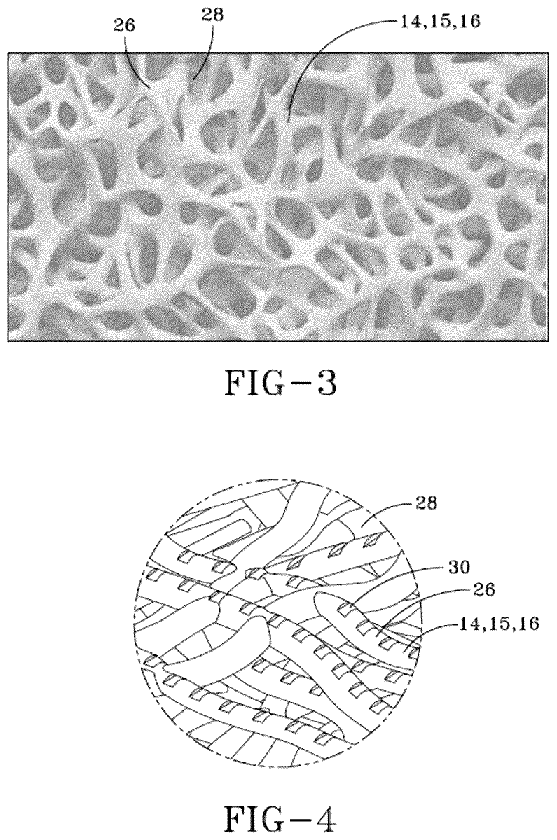

With reference to FIGS. 3 and 4, a portion of the exterior surface 14, 15, 16 is shown. This porous exterior surface can be along the surface of the first surface 14, second surface 16 or side surface 15 or all of these surfaces.

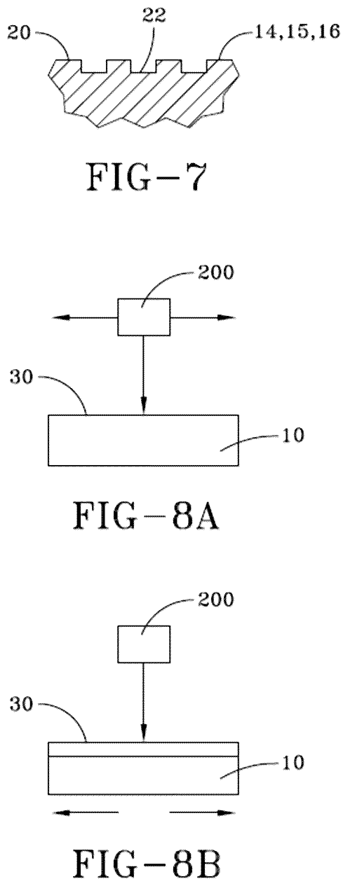

FIG. 7 is a simplified schematic outline of a portion of an exterior surface of an alternative embodiment, this exterior surface 14, 15, 16 has an undulating feature such that the exterior surface has protrusions 20 projecting outwardly slightly and channels or troughs 22 that are recessed slightly. These features create an undulating surface that enhances the ability of the device 10 to create space between the adjacent vertebrae when the device 10 is implanted. Additionally, this entire surface is then treated using laser etching to create nano channels 30 best shown in FIG. 4.

In FIG. 6, an exemplary laser etching machine 200 is illustrated that can be used to form nano channels. These nano channels 30 can be laid in a network 18 either in an organized uniform pattern or a random non-uniform pattern throughout the exterior surfaces 14, 15, 16. Ideally, these nano channels 30 are created at least along the first and second surfaces 14, 16 of the implant device 10. The nano channels 30 are small laser etched cuts that can be laid out along the entire exterior surfaces in a subtractive laser etching process. These nano channels 30 created by laser etching can be made either by moving the laser 200 about the exterior surface 14, 15, 16 of the implant device to form the nano channels 30 as shown in FIG. 8A; or the implant device 10 can be moved relative to the laser such that the nano channels 30 are laid onto the exterior surfaces 14, 15, 16 as shown in FIG. 8B. The nano channel features individually create an improved osteoinductive effect at the surface of the implant device 10. This means that the formation of new bone once implanted into the patient can be accelerated and the network 18 of nano channels 30 provide features that help assist in providing attachment locations for the new bone formation. This is an important feature that is provided in the current invention and is ideal in that it does not require smooth or flat exterior surfaces to form the channels which are effectively etched or burned into the exterior surface. The channels can be created as long as the path of the laser beam is unobstructed. As a result, even though the porous walls of exterior surfaces have slight undulations 20, 22 and openings, the network 18 of nano-channels 30 can be formed regardless of this and not limited to the topography of exterior surfaces commonly found in implant devices that are molded or otherwise have smooth exterior surfaces. In fact, the nano channels 30 can be found formed at varying depths where the openings allow the laser beam to pass. The nano channels preferably have a width and a depth of 10 nano meters or greater up to 1000 nano meters. These features are very small and unlike micro channel laser etching, the nano channels can be etched extremely quickly due to their small size.

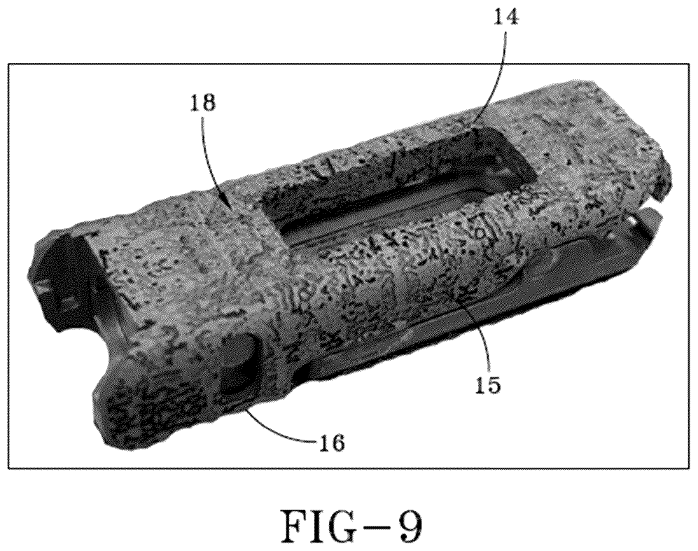

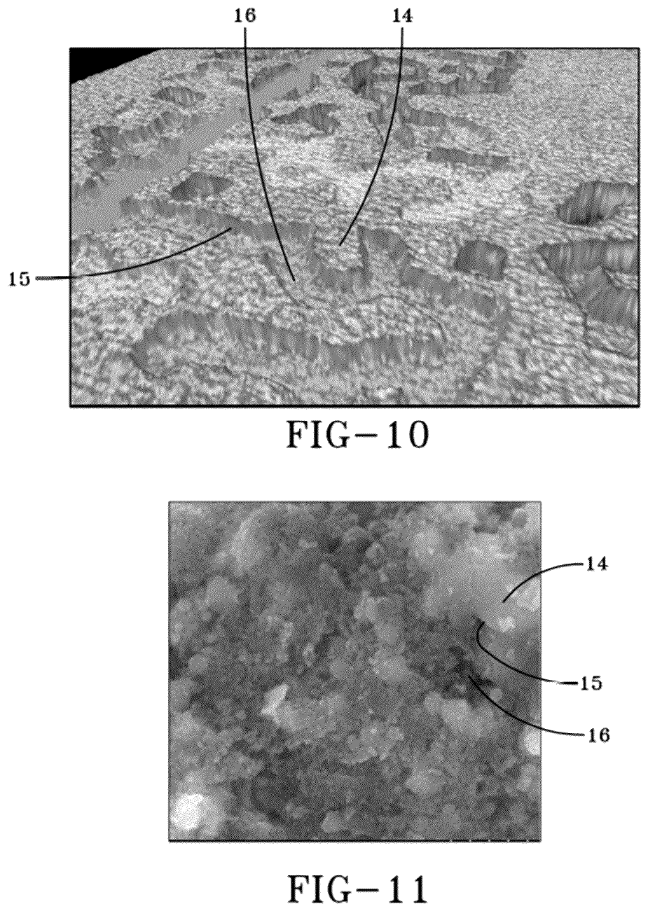

With reference to FIG. 9, a perspective view of an expandable spinal implant device 10 is shown having been made according to the methods described above. As shown, surfaces 14, 15, 16 have the porous feature for enhanced osteoinductivity for encouraging new bone to fuse to the device 10 after being installed in the disc space. The device 10, as shown, is in a partially expanded condition. The level of expansion can be raised to a higher amount or lowered to a closed position for insertion. The treated surfaces 14, 15, 16 are more refined than those shown in FIG. 1 and as can be seen in the enlarged view of FIG. 10 and magnified view of FIG. 11, illustrate the appearance of the trabecular bone mimicking surface using the nano-etching process. It is noted any implant device can be treated post manufacturing to create these surfaces on an existing implant device. Furthermore, the process can be used to form the surfaces on any number of implants where osteoinductive bone grown enhancement is desired. These can be bone fasteners, pedicle screws, cervical plates, spinal fusion cages and any bone contacting implant device that benefits from bone growth around the surface of the implant. The materials the implant device is made of can be any suitable implant material of metal, plastic or bone and the benefits of enhanced osteoinductivity can be achieved.

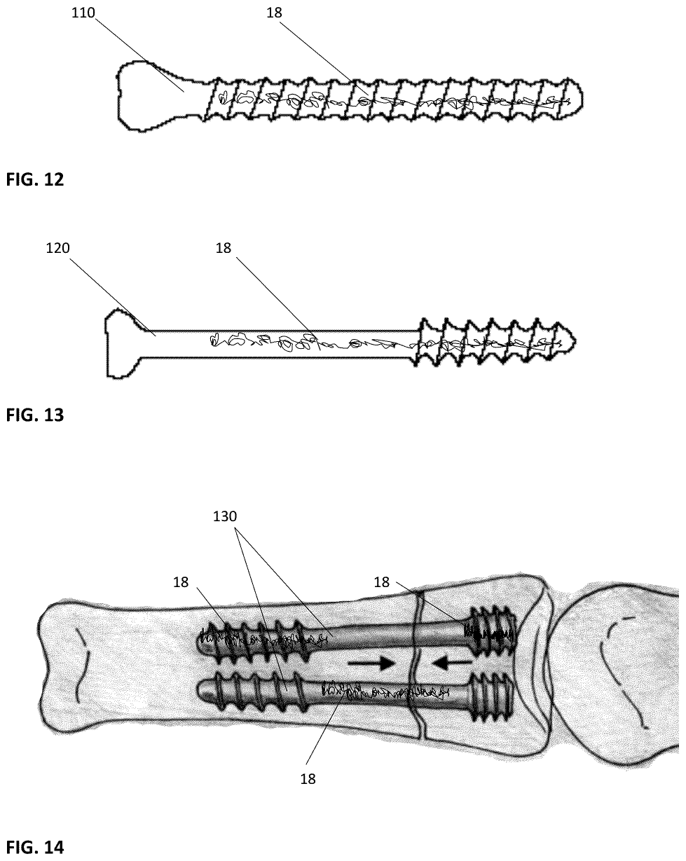

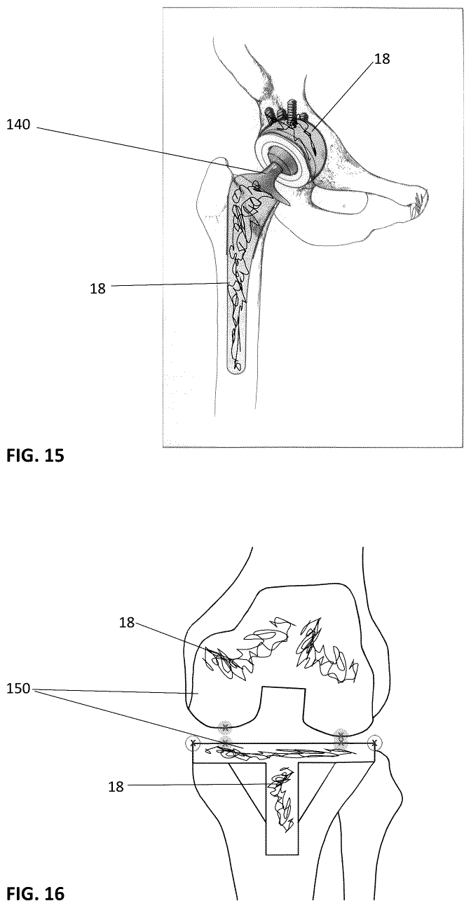

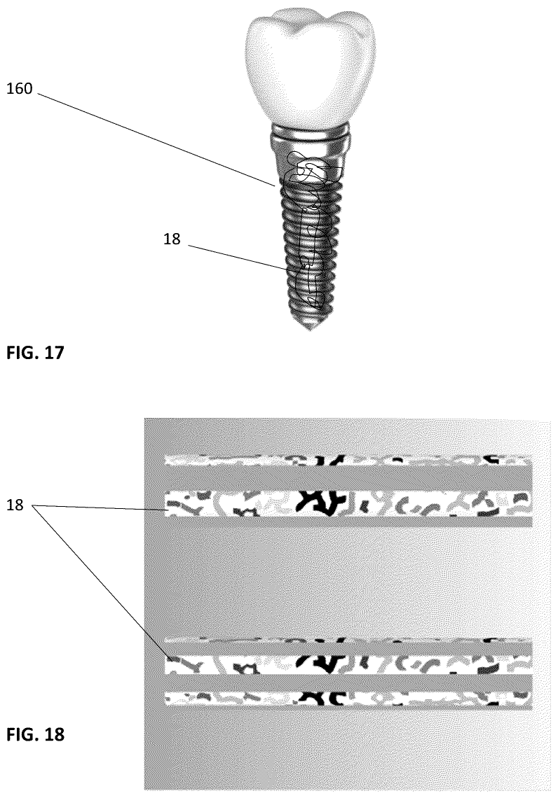

FIGS. 12-16 show various implants that can be produced by 3D printing or additive manufacturing and laser modified to produce the enhanced osteoinductivity of the present invention. FIG. 12 shows an exemplary cortical bone fastener or screw 110 with the network 18 of laser modified, etched or engraved features. FIG. 13 shows a second exemplary cancellous bone fastener or screw 120 with the network 18 of laser modified, etched or engraved features. FIG. 14 shows a third exemplary type of compression bone fastener or screw 130 used as a pair to join a fracture in a bone. FIG. 15 is a view of an exemplary orthopedic implant specifically a hip joint implant 140 with the laser modified network 18 on bone contacting surfaces. FIG. 16 is a view of another exemplary implant, specifically a knee replacement implant 150. FIG. 17 shows a dental implant 160 with the laser modified network 18 on bone contacting surfaces.

Laser modifications can be made using the exemplary methods and settings described herein. The laser modifications can be made on the unthreaded, threaded or both portions of the screw. Different treatment areas or options can be 4 of 8 surfaces or 6 of 12 surfaces as shown in FIG. 18 with a network 18 on an exemplary unthreaded portion of a screw. To determine how often the laser would rotate between engravings and by how much, surfaces could be treated with 45° partitions or split into 8 surfaces in order to maximize surface area engraved while minimizing excess movements. However, in order to keep 15° off-normal engraving, surfaces could be treated with 30° partitions or split into 12 surfaces. The below table shows a 3 strip treatment.

| | | | | |
|---|---|---|---|---|
| AREA | % AREA | TEXTURE | BLAST | TOTAL |

| | | | |
|---|---|---|---|
| NAME | [mm²] | [%] | [minutes] |

FOR A SINGLE SURFACE TREATMENT

| | | | | | |
|---|---|---|---|---|---|
| 1 of 8 | 7.79 | 12.50 | 0.97 | 0.13 | 1.10 |
| 1 of 12 | 5.19 | 8.33 | 0.63 | 0.08 | 0.72 |

FOR THREE (3) STRIPS OF TREATMENT

| | | | | | |
|---|---|---|---|---|---|
| 3 of 8 | 23.37 | 37.50 | 2.90 | 0.40 | 3.30 |
| 3 of 12 | 15.57 | 25.00 | 1.90 | 0.25 | 2.15 |

The method of making a spinal implant device or orthopedic device or bone implant device according to the present invention made by 3D printing and a post process with a laser etching process resulting in nanometer scale of surface structure that is biologically active in inducing bone growth. In addition, a 3D printing orthopedic or spinal device in which a surface pattern mimicking trabecular bone with arching structure mimicking trabecular bone formation that is created through the 3D printing process that either appears raised from the surface or recessed into the surface, either way it is made through the additive manufacturing process. Further, laser etched surface results in a nanometer scale structure that is active in bone growth formation. The laser etching results in a nanometer scale surface structure because the heat of the laser does not cause a significant melt at the surface that would remove material from the ablation from the nanometer scale of the structure rather than the laser heating it up so that it sears the surface through melting.

These and other aspects of the present invention are believed to greatly enhance the ability of the present device made by 3D printing and laser etching to provide an improved implant fusion device.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A method of making a spinal implant device or orthopedic device or bone implant device wherein the structure is produced through a 3D printing additive process, which is then further processed with a laser etching technology that results in a nanotechnology structure at the surface that facilitates bone attachment and growth.

2. The method of making a spinal implant device or orthopedic device or bone implant device of claim 1, wherein the 3D printing additive process creates a structure at the implant surface that mimics trabecular bone structure.

3. A spinal implant device or orthopedic device or bone implant device produced through a 3D printing additive process in a biocompatible material or materials that is further processed through a subtractive laser etching process that results in a surface or surfaces with nanometer-level structural elements.

4. The spinal implant device or orthopedic device or bone implant device of claim 3, wherein the 3D printing additive process results in surface features that mimic trabecular bone structure.

5. A method of making a spinal implant device or orthopedic device or bone implant device comprises the steps of:
fabricating an implant body structure using 3D printing to create the implant body structure;
additively building the body structure having a superior load bearing surface and an inferior load bearing surface and a wall structure; and
wherein the body structure has at least a portion of the body structure having a plurality of interconnected struts forming porous walls with openings extending inwardly from an exterior surface to a depth of 1.0 mm or greater forming a porous portion with a void volume to solid mass volume mimicking trabecular bone.

6. The method of making a spinal implant device or orthopedic device or bone implant device of claim 5, wherein the average or nominal ratio of void volume to mass volume in the porous portion is in the range of 65 percent or more, preferably 75 percent replicating that of trabecular bone in an adult male.

7. The method of making a spinal implant device or orthopedic device or bone implant device of claim 6, wherein the struts of the porous walls are curved or arch shaped with openings communicating with adjacent walls.

8. The method of making a spinal implant device or orthopedic device or bone implant device of claim 7, wherein the porous portion of the implant body structure extends at least partially across the implant body structure to the exterior surfaces forming conduits for fluid passage throughout the device.

9. The method of making a spinal implant device or orthopedic device or bone implant device of claim 8, wherein the curved or arch shaped struts of the porous walls create a load bearing capacity to withstand vertical loads without collapsing.

10. The method of making a spinal implant device or orthopedic device or bone implant device of claim 5, wherein the implant fusion device has the superior load bearing surface and the inferior load bearing surface, each load bearing surface having nano channels etched on exposed surfaces.

11. The method of making a spinal implant device or orthopedic device or bone implant device of claim 6, wherein the nano channels are made into a network of features in either a random pattern or an organized pattern.

12. The method of making a spinal implant device or orthopedic device or bone implant device of claim 11, wherein the nano channels are formed by emitting laser beams unobstructed to surfaces within the path of the laser beams.

13. The method of making a spinal implant fusion device of claim 11, wherein the implant body structure is stationary and a laser moves about the implant body structure to create the network of features or wherein a laser is stationary and the implant body structure moves relative to the laser to create the network of features.

14. A method of making a spinal implant device or orthopedic device or bone implant device comprises the steps of:
providing an implant body structure; and
laser etching nano channels on at least a portion of an exterior surface or surfaces of the implant body structure, the nano channels creating new bone growth attachment features to enhance osteoinductivity of the spinal implant fusion device.

15. The method of making a spinal implant device or orthopedic device or bone implant device of claim 14, wherein the laser etched nano channels are made into a network of features in either a random pattern or an organized pattern.

16. The method of making a spinal implant device or orthopedic device or bone implant device of claim 15, wherein the laser etching is formed by emitting laser beams unobstructed to the exterior surfaces.

17. The method of making a spinal implant device or orthopedic device or bone implant device of claim 16, further comprises the step of moving a laser about the implant body structure to create the network of features.

18. The method of making a spinal implant device or orthopedic device or bone implant device of claim 16, further comprises the step of moving the implant body structure about a laser to create the network of features.

19. A method of making a spinal implant device or orthopedic device or bone implant device comprises the steps of:
fabricating an implant body structure using 3D printing to create the implant body structure;
additively building the body structure having a superior load bearing surface and an inferior load bearing surface and a wall structure;
wherein the body structure has at least a portion of the body structure having a plurality of walls with openings extending inwardly from an exterior surface to a depth of 1.0 mm or greater forming a porous portion with a void volume to solid mass volume mimicking trabecular bone; and
laser etching nano channels on at least a portion of the exterior surface or surfaces of the implant body structure, the nano channels creating new bone growth attachment features to enhance osteoinductivity of the spinal implant fusion device.

20. The method of making a spinal implant device or orthopedic device or bone implant device of claim 19, wherein the average or nominal ratio of void volume to mass volume of the porous portion is in the range of 65 percent or more, more preferably 75 percent replicating that of trabecular bone in an adult male.

21. The method of making a spinal implant device or orthopedic device or bone implant device of claim 19, wherein one or more of the plurality of walls is curved or arch shaped with openings communicating with adjacent walls.

22. The method of making a spinal implant device or orthopedic device or bone implant device of claim 19, wherein the porous portion of the implant body structure extends across the implant body structure from the exterior surfaces inwardly forming conduits for fluid passage throughout the porous portion of the implant body structure.

23. The method of making a spinal implant device or orthopedic device or bone implant device of claim 22, wherein the curved or arch shaped walls create a load bearing capacity to withstand vertical loads without collapsing.

24. The method of making a spinal implant device or orthopedic device or bone implant device of claim 19, wherein the implant fusion device has the superior load bearing surface and the inferior load bearing surface having nano channels etched on exposed surfaces.

25. The method of making a spinal implant device or orthopedic device or bone implant device of claim 24, wherein the nano channels are made into a network of features in either a random pattern or an organized pattern.

26. The method of making a spinal implant device or orthopedic device or bone implant device of claim 19, wherein the nano channels are formed by emitting laser beams unobstructed to surfaces within the path of the laser beams.

27. The method of making a spinal implant device or orthopedic device or bone implant device of claim 25, wherein the implant body structure is stationary and a laser moves about the implant body structure to create the network of features.

28. The method of making a spinal implant device or orthopedic device or bone implant device of claim 25, wherein a laser is stationary and the implant body structure moves relative to the laser to create the network of features.

29. The spinal implant device or orthopedic device or bone implant device of claim 3, wherein the device is one of a spinal implant fusion device, a bone screw or fastener, a dental implant, a hip implant, or a knee implant.
